(19) United States
(12) Patent Application Publication
(10) Pub. No.: US 2020/0393451 A1
(43) Pub. Date: Dec. 17, 2020

(54) MULTIPLEXED TURBIDIMETRIC IMMUNOASSAYS TO MONITOR ENVIRONMENTAL CONTAMINATION

(71) Applicant: Becton, Dickinson and Company, Franklin Lakes, NJ (US)

(72) Inventors: Christian Sandmann, Wayne, NJ (US); Brian Dwyer, San Diego, CA (US)

(21) Appl. No.: 16/898,250

(22) Filed: Jun. 10, 2020

Related U.S. Application Data
(60) Provisional application No. 62/861,236, filed on Jun. 13, 2019.

(51) Int. Cl. G01N 33/543; G01N 1/28; G01N 21/51; G01N 21/59

(52) U.S. Cl. CPC G01N 33/54313; G01N 21/59; G01N 21/51; G01N 1/28

(57) ABSTRACT

Provided herein are methods and kits for detecting environmental contaminants using turbidimetric assays. Certain embodiments of the present disclosure are related to methods of obtaining a sample from a test surface and contacting the collected sample with a turbidimetric composition for quantification of an analyte in the sample.

### CROSS REFERENCE TO RELATED APPLICATION

This application claims the benefit of U.S. Provisional Application No. 62/861,236, filed Jun. 13, 2019, which is hereby incorporated by reference in its entirety.

### FIELD

The methods, systems, and kits disclosed herein are directed to environmental contaminant testing, and, more particularly, to methods, systems, and kits for detecting the presence and/or quantity of antineoplastic agents on a test surface using a turbidimetric assay.

### BACKGROUND

Antineoplastic drugs are used to treat cancer, and are often found in a small molecule (like fluorouracil) or antibody format (like Rituximab). Detection of antineoplastic drugs is critical for determining if there is contamination or leakage where the drugs are used and/or dispensed, such as hospital and pharmacy areas.

The nature of antineoplastic drugs make them harmful to healthy cells and tissues as well as the cancerous cells. Precautions should be taken to eliminate or reduce occupational exposure to antineoplastic drugs for healthcare workers. Pharmacists who prepare these drugs and nurses who may prepare and administer them are the two occupational groups who have the highest potential exposure to antineoplastic agents. Additionally, physicians and operating room personnel may also be exposed through the treatment of patients, as patients treated with antineoplastic drugs can excrete these drugs. Hospital staff, such as shipping and receiving personnel, custodial workers, laundry workers and waste handlers, all have the potential to be exposed to these drugs during the course of their work. The increased use of antineoplastic agents in veterinary oncology also puts these workers at risk for exposure to these drugs.

### SUMMARY

Described herein are systems, methods, and kits for environmental analysis of an analyte of interest. In some embodiments, the analyte of interest is an antineoplastic agent, such as cyclophosphamide, docetaxel, fluorouracil, ifosfamide, imatinib, or paclitaxel.

Some embodiments provided herein relate to methods of quantifying an analyte of interest in a sample. In some embodiments, the methods include obtaining or having obtained a sample having or suspected of having an analyte of interest. In some embodiments, the sample is obtained from a test surface. In some embodiments, the methods further include contacting the sample with a turbidimetric composition and measuring a turbidity of the composition. In some embodiments, the measured turbidity corresponds to a quantity of analyte of interest in the sample. In some embodiments, the turbidimetric composition includes a detection particle comprising immobilized analyte, and a binding reagent that specifically binds analyte of interest. In some embodiments, obtaining the sample form the test surface includes collecting the analyte of interest from the test surface, placing the collected analyte of interest in a test solution, removing an aliquot of the test solution, and transferring the aliquot to an assay vessel comprising the turbidimetric composition. In some embodiments, obtaining the sample from the test surface includes wiping the test surface with an absorbent material to collect the analyte of interest from the test surface, inserting the absorbent material into a collection container, the collection container comprising a second volume of the buffer solution, sealing the collection container to isolate the first and second volumes of the buffer solution within the collection container, agitating the collection container to release at least some of the collected analyte of interest into the buffer solution, and transferring a third volume of the buffer solution from the collection container to an assay vessel comprising the turbidimetric composition. In some embodiments, the absorbent material is moistened with a first volume of a buffer solution configured to lift the analyte of interest from the test surface. In some embodiments, the absorbent material is coupled to an elongate handle. In some embodiments, the assay vessel is a cuvette, a tube, a microtiter plate, or a microfluidic device. In some embodiments, the detection particle aggregates with other detection particles present in the turbidimetric composition in the absence of analyte of interest, thereby increasing turbidity of the turbidimetric composition. In some embodiments, increased concentrations of analyte of interest in the sample decreases aggregation of the detection particles, thereby decreasing turbidity of the turbidimetric composition. In some embodiments, the turbidity of the turbidimetric composition is measured using a detection device that measures turbidity of the turbidimetric composition. In some embodiments, the detection device includes control electronics configured to analyze signals representative of analyte concentration and configured to determine the quantity of the analyte of interest in the sample based on turbidity of the turbidimetric composition. In some embodiments, the measured turbidity of the turbidimetric composition is compared to a standard curve to determine the quantity of the analyte of interest. T In some embodiments, the analyte of interest is a hazardous contaminant. In some embodiments, the analyte of interest is an antineoplastic agent. In some embodiments, the analyte of interest is cyclophosphamide, docetaxel, fluorouracil, ifosfamide, imatinib, or paclitaxel. In some embodiments, the test surface is a surface at a hospital or pharmacy. In some embodiments, the surface is a counter, a cabinet, an instrument, a bed, a chair, a table, a floor, a window, a toilet, or a wall. In some embodiments, the analyte of interest is not obtained from a plasma sample. In some embodiments, the detection particle is a nanoparticle. In some embodiments, the detection particle comprises organic or inorganic polymers, liposomes, latex, phospholipid vesicles, or lipoproteins. In some embodiments, the binding reagent is an antibody or fragment thereof that specifically binds the analyte of interest. In some embodiments, the analyte of interest is present in an amount ranging from 10 fg/mL to 10 μg/mL, such as 10, 15, 20, 25, 30, 35, 40, 50, 60, 70, 80, 90, 100, 200, 300, 400, 500, 600, 700, 800, 900, or 1000 fg/mL, or 1, 2, 3, 4, 5, 6, 7, 8, 9, 10, 15, 20, 25, 30, 35, 40, 50, 60, 70, 80, 90, 100, 200, 300, 400, 500, 600, 700, 800, 900, or 1000 pg/mL, or 1, 2, 3, 4, 5, 6, 7, 8, 9, 10, 15, 20, 25, 30, 35, 40, 50, 60, 70, 80, 90, 100, 200, 300, 400, 500, 600, 700, 800, 900, or 1000 ng/mL, or 1, 2, 3, 4, 5, 6, 7, 8, 9, or 10 μg/mL. In some embodiments, the detection particle is present in an amount ranging from 10 to 100,000,000 particles per mL, such as 10, 100, 1000, 2000, 3000, 4000, 5000, 6000, 7000, 8000, 9000, 10,000, 15,000, 20,000, 30,000, 40,000, 50,000, 60,000, 70,000, 80,000, 90,000, 100,000, 150,000, 200,000, 250,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, or 100,000,000. In some embodiments, the immobilized analyte is present on the detection particle in an amount ranging from 1 analyte per detection particle to 100,000 analyte per detection particle, such as 1, 2, 3, 4, 5, 6, 7, 8, 9, 10, 15, 20, 25, 30, 35, 40, 45, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, or 100,000 analytes per detection particle. In some embodiments, the binding reagent is present in an amount ranging from 0.001 μg/mL to 10 μg/mL, such as 0.001, 0.005, 0.01, 0.05, 0.1, 0.5, 1, 2, 3, 4, 5, 6, 7, 8, 9, or 10 μg/mL. In some embodiments, measuring the turbidity of the turbidimetric composition is performed at a wavelength ranging from 300 to 700 nm, such as 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, or 700 nm.

Some embodiments provided herein relate to kits for determining a quantity of an analyte of interest obtained from a test surface. In some embodiments, the kits include an analyte collection device for collecting the analyte of interest from a test surface, a turbidimetric composition, and an assay vessel. In some embodiments, the turbidimetric composition includes a detection particle comprising immobilized analyte, and a binding reagent that specifically binds analyte of interest. In some embodiments, the analyte collection device includes a buffer solution configured to lift the analyte of interest from the test surface when the buffer solution is applied to the test surface, an absorbent material configured to absorb at least a portion of the buffer solution and to contact the test surface to collect the analyte of interest, and a collection container having an interior volume dimensioned to encase the absorbent material and the buffer solution. In some embodiments, the detection particle is present in an amount ranging from 10 to 100,000,000 particles per mL, such as 10, 100, 1000, 2000, 3000, 4000, 5000, 6000, 7000, 8000, 9000, 10,000, 15,000, 20,000, 30,000, 40,000, 50,000, 60,000, 70,000, 80,000, 90,000, 100,000, 150,000, 200,000, 250,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, or 100,000,000. In some embodiments, the immobilized analyte is present on the detection particle in an amount ranging from 1 analyte per detection particle to 100,000 analyte per detection particle, such as 1, 2, 3, 4, 5, 6, 7, 8, 9, 10, 15, 20, 25, 30, 35, 40, 45, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, or 100,000 analytes per detection particle. In some embodiments, the binding reagent is present in an amount ranging from 0.001 μg/mL to 10 μg/mL, such as 0.001, 0.005, 0.01, 0.05, 0.1, 0.5, 1, 2, 3, 4, 5, 6, 7, 8, 9, or 10 μg/mL. In some embodiments, the assay vessel is a cuvette, a tube, a microtiter plate, or a microfluidic device.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIGS. 1A-1B schematically depict turbidimetric interactions. FIG. 1A schematically depicts an example turbidimetric assay, wherein no analyte is present in the sample, resulting in increased aggregation, and increased turbidity. FIG. 1B schematically depicts an example turbidimetric assay, wherein analyte is present in the sample, resulting in decreased aggregation, and decreased turbidity. The representations of molecules and their binding interactions shown in FIGS. 1A and 1B are representative only, and are not necessarily intended to depict actual binding interactions that take place between molecules.

FIG. 2 graphically illustrates an example turbidimetric reading as a function of analyte concentration, where the degree of turbidity is inversely related to the quantity of analyte.

FIG. 3 illustrates a schematic block diagram of an example method for measuring quantity of an analyte obtained from a test surface using a turbidimetric assay.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein. All references cited herein are expressly incorporated by reference herein in their entirety and for the specific disclosure referenced herein.

Antineoplastic drugs are antiproliferative. In some cases they affect the process of cell division by damaging DNA and initiating apoptosis, a form of programmed cell death. While this can be desirable for preventing development and spread of neoplastic (e.g., cancerous) cells, antineoplastic drugs can also affect rapidly dividing non-cancerous cells. As such, antineoplastic drugs can suppress healthy biological functions including bone marrow growth, healing, hair growth, and fertility, to name a few examples.

Studies have associated workplace exposures to antineoplastic drugs with health effects such as skin rashes, hair loss, infertility (temporary and permanent), effects on reproduction and the developing fetus in pregnant women, increased genotoxic effects (e.g., destructive effects on genetic material that can cause mutations), hearing impairment and cancer. The hazardous occupational exposure to antineoplastic drugs thus presents a serious health burden to care providers and workers involved in handling, preparation, or administering the drugs, as well as to those cleaning objects that have come into contact with the drugs. These health risks are influenced by the extent of the exposure and the potency and toxicity of the hazardous drug. Although the potential therapeutic benefits of hazardous drugs may outweigh the risks of such side effects for ill patients, exposed health care workers risk these same side effects with no therapeutic benefit. Further, it is known that exposures to even very small concentrations of antineoplastic drugs may be hazardous for workers who handle them or work near them, and for known carcinogenic agents there is no safe level of exposure.

Environmental sampling can be used to determine the level of workplace contamination by antineoplastic agents. Sampling and decontamination of contaminated areas is complicated, however, by a lack of quick, inexpensive methods to first identify these areas and then determine the level of success of the decontamination. Although analytical methods are available for testing for the presence of antineoplastic drugs in environmental samples, these methods require shipment to outside labs, delaying the receipt of sampling results.

In addition, some systems determine clinical levels of antineoplastics in human plasma using automated clinical analyzers as an aid in the management of antineoplastic therapy, such in for management of paclitaxel therapy, fluorouracil therapy (including 5-fluorouracil therapy), or docetaxel therapy, for example, as described in U.S. Pat. Nos. 7,175,993, 7,205,116, 7,459,281, and 8,076,097, each of which is incorporated by reference herein in its entirety and for the specific disclosure referenced herein. These systems include turbidimetric systems for analyzing antineoplastic levels in a blood or serum sample of a subject being treated with an antineoplastic.

Embodiments of the systems, methods, and kits provided herein are related to determining a quantity of an analyte of interest obtained or having obtained from a test surface using a turbidimetric assay.

As used herein, the term “analyte of interest” has its ordinary meaning as understood in light of the specification, and refers to an agent to be detected that is obtained from an environmental source. For example, the analyte of interest may be a hazardous contaminant obtained from an environmental source. Any analyte that may be found or suspected of being found, or obtained in an environment may be an analyte of interest. The analyte of interest may be obtained from any surface found within any environment where an analyte of interest is typically found or suspected of being found. For example, the analyte of interest may be an analyte that is found in a hospital, health care, clinical, research, pharmacy, forensic, or industrial environment. The analyte of interest may be obtained from a surface in a hospital, health care facility, clinic, research facility, or pharmacy, such as from a surface of a bench, desk, counter, cabinet, wall, floor, window, instrument, table, chair, toilet or bed found within the environment. The analyte of interest may be obtained by the user that measure the quantity of analyte of interest, or the analyte of interest may be obtained by an upstream user, who then provides the analyte of interest to the user who measured the analyte of interest in the sample.

In some embodiments, the analyte of interest is not obtained from a biological sample directly obtained from a patient. For example, the analyte of interest is not obtained from a human blood sample, a human plasma sample, a human urine sample, or from any other biological sample directly obtained from a patient. In some embodiments, the sample is obtained from a biological sample obtained indirectly from a patient, for example blood or urine found on a surface in a hospital or treatment room (e.g. a bathroom surface).

Any analyte of interest for which it may be desirable to determine a quantity in an environment may be analyzed, particularly an analyte of interest that poses a health risk in an environment, and which it would be desirable to determine the presence and concentration of in a given environment. In some embodiments, the analyte of interest is a drug (including toxic drugs, such as a hazardous drug including antineoplastic agents), a steroid, a toxin, a pesticide, a biowarfare agent, or any other detectable analyte of interest.

In some embodiments, the analyte of interest is an antineoplastic agent. As used herein the term “antineoplastic agent” has its ordinary meaning as understood in light of the specification, and refers to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents. In some embodiments, the analyte of interest is afatinib, aflibercept, alemtuzumab, alitretinoin, altretamine, anagrelide, arsenic trioxide, asparaginase, axitinib, azacitidine, BCG vaccine, bendamustine, bevacizumab, bexarotene, bosutinib, bleomycin, bortezomib, busulfan, cabazitaxel, capecitabine, carboplatin, carmofur, carmustine, cetuximab, chlorambucil, cisplatin, cladribine, clofarabine, crizotinib, cyclophosphamide, cytarabine, dacarbazine, dactinomycin, dasatinib, daunorubicin, decitabine, denileuikin diftitox, denosumab, docetaxel, doxorubicin, epirubicin, erlotinib, estramustine, etoposide, everolimus, floxuridine, fludarabine, fluorouracil, fotemustine, gefitinib, gemcitabine, gemtuzumab ozogamicin, hydroxycarbamide, ibritumomab tiuxetan, idarubicin, ifosfamide, imatinib, ipilimumab, irinotecan, isotretinoin, ixabepilone, lapatinib, lenalidomide, lomustine, melphalan, mercaptopurine, methotrexate, mitomycin, mitoxantrone, nedaplatin, nelarabine, nilotinib, nivolumab, ofatumumab, oxaliplatin, paclitaxel, panitumumab, panobinostat, pazopanib, pembrolizumab, pemetrexed, pentostatin, pertuzumab, pomalidomide, ponatinib, procarbazine, raltitrexed, regorafenib, rituximab, romidepsin, ruxolitinib, sorafenib, streptozotocin, sunitinib, tamibarotene, tegafur, temozolomide, temsirolimus, teniposide, thalidomide, tioguanine, topotecan, tositumomab, trastuzumab, tretinoin, valproate, valtubicin, vandetanib, vemurafenib vinblastine, vincristine, vindesine, vinflunine, vinorelbine, or vorinostat, or any derivative, conjugate, or analogue thereof.

In some embodiments, the analyte of interest is a steroid. In some embodiments, the steroid is a glucocorticoid and includes, for example, hydroxycortisone, cortisone, desoxycorticosterone, fludrocortisone, betamethasone, beclometasone, dexamethasone, prednisolone, prednisone, methylprednisolone, paramethasone, triamcinolone, flumethasone, fluocinolone, fluocinonide, fluprednisolone, halcinonide, flurandrenolide, meprednisone, medrysone, clobetasol, and esters, mixtures, analogues, or derivatives thereof.

In some embodiments, the analyte of interest is a toxin. As used herein, the term “toxin” has its ordinary meaning as understood in light of the specification, and refers to an agent exhibiting poisonous properties to living cells or organisms. Toxins may include, for example, small molecules, peptides, or proteins, and may include biotoxins or environmental toxins.

In some embodiments, the analyte of interest is a pesticide. As used herein, the term “pesticide” has its ordinary meaning as understood in light of the specification, and refers to an agent or agents that controls pests. Pesticides may include, for example, algicides, antifouling agents, antimicrobials, attractants, biopesticides, biocides, disinfectants, fungicides, fumigants, herbicides, insecticides, miticides, microbial pesticides, molluscicides, nematicides, ovicides, pheromones, repellents, or rodenticides.

In some embodiments, the analyte of interest is a biowarfare agent, which may include, for example, a biological toxin, an infectious agent, such as a bacteria, virus, or fungi, or other agent intended for killing or harming biological organisms, such as humans, animals, or plants.

In some embodiments, the analyte of interest is obtained from a test surface using a collection device. As described herein, a test surface is any surface where any analyte of interest as described herein may be obtained, or where any analyte of interest may be suspected of being found. For example, the test surface may be a surface of any object found in a hospital, health care facility, clinic, research facility, or pharmacy. In some embodiments, the test surface is a surface of a bench, desk, counter, cabinet, wall, floor, window, instrument, table, chair, toilet or bed found within the environment.

A collection device refers to a device that is used for obtaining an analyte of interest from a test surface. In some embodiments, the collection device is a collection device as described in PCT Publication No. WO 2019/060269, which is incorporated by reference herein in its entirety and for the specific disclosure referenced herein.

In some embodiments, the collection device is a collection kit that includes a buffer solution configured to solubilize, transport, or remove an analyte of interest from a test surface when the buffer solution is applied to the test surface, and an absorbent swab material configured to absorb at least a portion of the buffer solution and to contact the test surface to collect the analyte of interest. In some embodiments, the absorbent swab material is coupled to a first end of a handle. In some embodiments, the handle has a second end spaced apart from the first end, and an elongate length extending therebetween. In some embodiments, the collection kit further includes a fluid-tight container having an interior volume dimensioned to encase the handle and the absorbent swab material and the buffer solution, the container having a nozzle including an orifice sized to provide controlled release of a volume of the buffer solution from the interior volume.

In some embodiments, the absorbent swab material can be constructed from a material having desired adsorbent efficiency and shedding efficiency for detecting trace amounts of an analyte of interest, and is provided on a handle having sufficient length so that the user can swab a surface without physically contacting the test surface or the absorbent swab material. In some embodiments, a liquid, for example a buffer solution, can be provided within the container so that the user removes a pre-wetted absorbent swab material to swipe the surface. In some embodiments, the user sprays the surface with a liquid and collects this liquid with the absorbent swab material.

In some embodiments, the collection kit further includes a demarcation guide specifying an area of the test surface to be tested for contamination by the hazardous contaminant. The collection kit can further include a template, guide, or instructions to delineate a specific dimensional area for testing. In order to obtain an accurate test result for contaminants that are hazardous even in trace amounts, a precise method of marking (demarcation) and then performing the sampling procedure (for example, to sample all of the demarcated area and only the demarked area) can be an important step to ensure accurate determination of presence and quantity of analyte of interest. Several factors may be key to obtaining an accurate measurement of concentration of analyte of interest, as given in the following formula:

$$C = \frac{∝ * A * {η}_{p} * {η}_{e}}{{V}_{b}}$$

where C is the concentration, α is the contamination surface density (ng/ft²), A is the surface area swabbed and tested, ηₚ is the pick-up efficiency, ηₑ is the extraction from the swab density, and V_{b} is the fluid volume of the buffer solution used to help extract and carry the contamination to the test strip. A goal of the described testing can be to have a high concentration signal with low variability. Excessive “noise” or variation in the variables may cause the test to either give false positive or false negative results. Collection devices described herein can include mechanisms and/or instructions to users to assist in reducing the variation of each term in the above concentration equation.

In some embodiments, after swabbing the test surface, the user places the absorbent swab material into the container and the handle forms a liquid-tight seal when engaged with the container. The handle can additionally lock to the container. The container can contain a buffer or diluent solution used as an agent to help remove the analyte of interest embedded on the absorbent swab material into the fluid of the container. The container advantageously prevents liquid from spilling and contaminating surfaces or users, but provides for controlled release of fluid to a detection system. Non-limiting examples of such systems are referred to herein as “open system contaminant collection devices” and “open system detection devices.” In some embodiments, the container comprises an open end having an aperture into the interior volume, and a releasable portion of the container including an attachment mechanism configured to releasably couple to the container over the open end to provide a fluid-tight seal with the interior volume of the container with the handle and the absorbent swab material and the buffer solution sealed within the interior volume, the nozzle, and a cap releasably coupled to the nozzle.

The described collection device, absorbent swab material, and buffer solutions can be configured to collect trace amounts of analytes of interest. It will be appreciated that the described systems can be adapted to collect and detect quantities of various analytes of interest, including for example, a drug (including toxic drugs, such as a hazardous drug including antineoplastic agents), a steroid, a toxin, a pesticide, a biowarfare agent, or any other detectable analyte of interest. Further, the disclosed systems can be used in hospital, health care, clinical, research, pharmacy, forensic, industrial, and other settings.

In some embodiments, after obtaining the analyte of interest from the test surface using a collection device as described herein, the analyte of interest is contacted with an assay composition. In some embodiments, the assay composition is contained in an assay vessel. In some embodiments, the assay vessel is any vessel that is capable of containing a liquid composition and in which an amount of analyte of interest may be detected. In some embodiments, the assay vessel is a cuvette, a tube, a microtiter plate, or a microfluidic device.

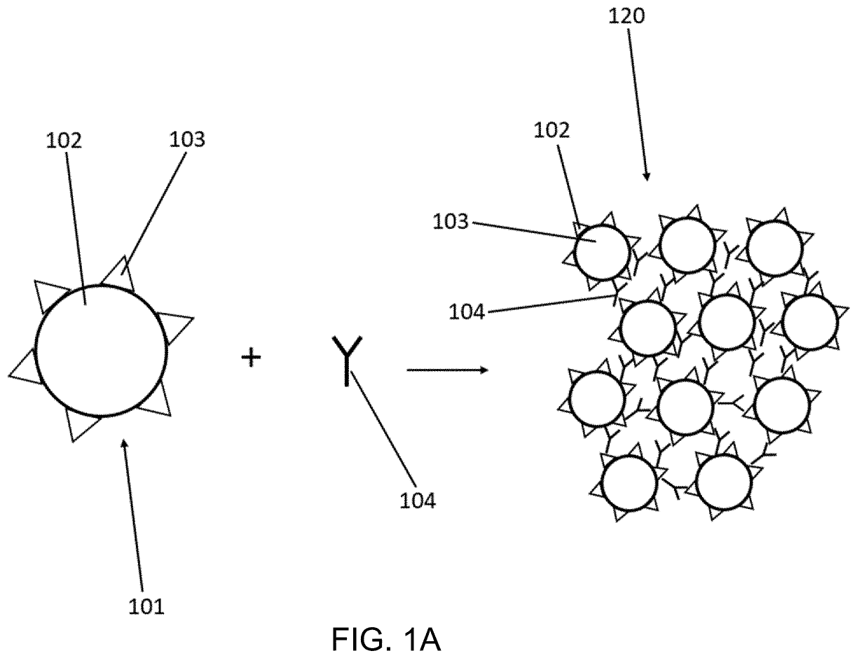

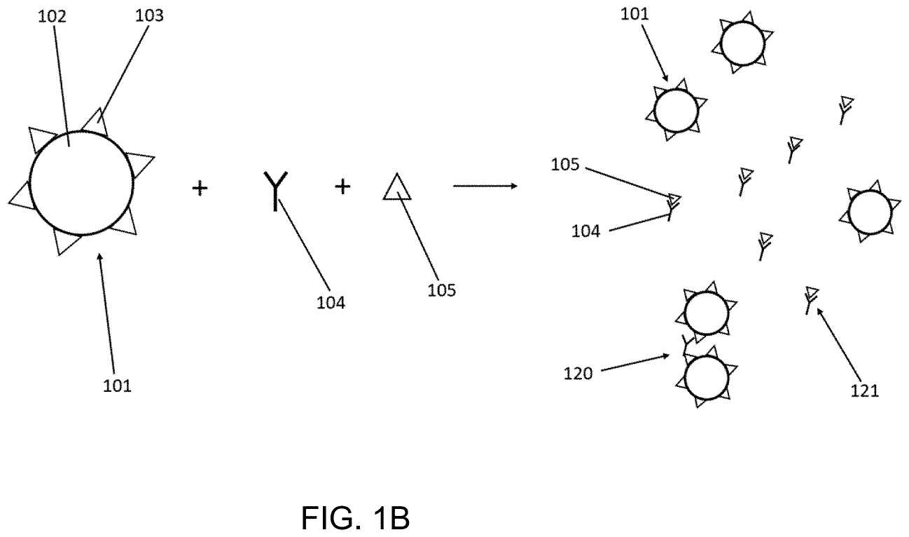

In some embodiments, the assay composition is a turbidimetric composition. In some embodiments, the assay composition includes a detection particle comprising immobilized analyte and a binding reagent that specifically binds analyte of interest. With reference to FIG. 1A, a detection particle 102 comprising immobilized analyte 103 is shown as a complex 101. The detection particle 102 may be any particle to which analyte 103 may be immobilized, and which result in a turbid composition upon aggregation. In some embodiments, the detection particle 102 is organic or inorganic polymers, liposomes, latex, phospholipid vesicles, or lipoproteins. In some embodiments, the detection particle 102 is a latex bead. The size of the detection particle 102 may be any size such that a single detection particle 102 is not detected alone, but that aggregation of many detection particles 102 results in a turbid composition that can be detected using a reader device. Thus, for example, the detection particle 102 is a size ranging from about 5 nm to about 1,000 nm, such as 5, 10, 15, 20, 25, 30, 35, 40, 45, 50, 60, 70, 80, 90, 100, 150, 200, 250, 300, 400, 500, 600, 700, 800, 900, or 1,000 nm, or a size within a range defined by any two of the aforementioned values.

The complex 101 further includes an analyte 103 that is immobilized to the detection particle 102. The immobilized analyte 103 may be the same analyte 103 as the analyte of interest 105, or the immobilized analyte 103 may be a different analyte 103 as the analyte of interest 105. The immobilized analyte 103 contains a recognition moiety to which a binding reagent 104 specifically binds. The binding reagent 104 is capable of specifically binding to the immobilized analyte 103 and to an analyte of interest 105, if present in the sample. In some embodiments, the binding reagent 104 will contain multiple recognition sites, such that the binding reagent 104 is capable of binding multiple immobilized analytes 103. Thus, with reference to FIG. 1A, when a composition comprising the complex 101 is contacted with a composition comprising the binding reagent 104, the binding reagent 104 specifically binds to immobilized analyte 103 on one complex 101 and also specifically binds to immobilized analyte 103 on an additional complex 101, thereby aggregating the complexes. As shown in FIG. 1A, in the absence of analyte of interest 105, the combination of the complex 101 comprising a detection particle 102 and immobilized analyte 103 with a binding reagent 104 results in a complex aggregation 120. Aggregation of the complexes increased the turbidity of the composition, and the degree of turbidity is measured.

As shown in FIG. 1B, when a composition comprising complex 101 that comprises a detection particle 102 with immobilized analyte 103 is contacted with a composition comprising the binding reagent 104, and where analyte of interest 105 is present, the analyte of interest 105 competes with the complex 101 for the binding reagent 104, such that at least some of the binding reagent 104 specifically binds to analyte of interest 105, thereby decreasing the amount of available binding reagent 104 that can cause aggregation of the complexes 101. Although complex aggregation 120 may still be present in the presence of analyte of interest 105, the degree of complex aggregation 120 decreases due to the formation of analyte of interest-binding reagent complex 121. This results in decreased turbidity of the composition as compared to the composition having no analyte of interest 105 present. Thus, the degree of turbidity in a composition is inversely related to a quantity of analyte of interest 105 present in the solution.

The depiction of the aggregation of molecules as shown in FIGS. 1A and 1B is shown with respect to the use of antibodies as the binding reagent 104. The binding interactions as shown in FIGS. 1A and 1B are illustrative, and are not intended to depict the actual binding interactions that take place between the binding reagent 104 and the immobilized analyte 103 on the detection particle 102.

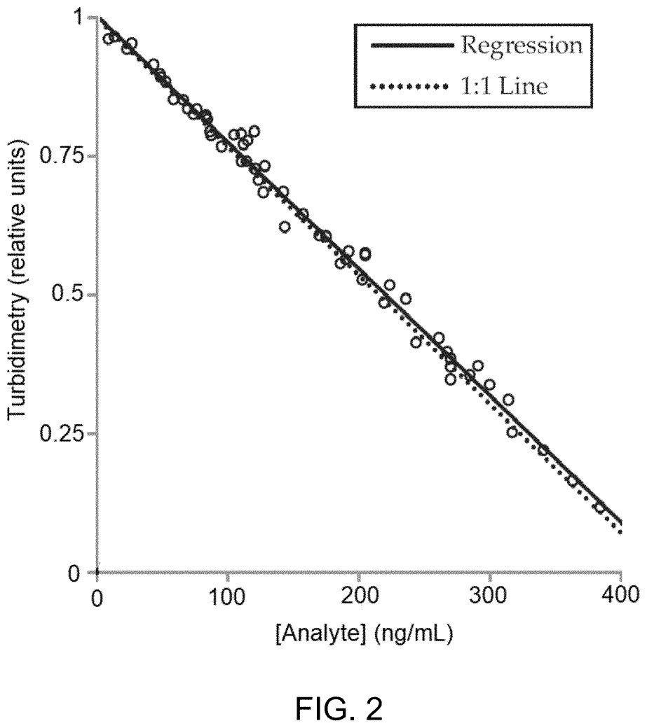

As shown in FIG. 2, in the absence of analyte of interest, the relative measurement of turbidity is at a maximum value. As the concentration of analyte of interest increases, the measurement of turbidity decreases, which is due to the analyte of interest competing with the complex for binding reagent. Because less binding reagent is available with increasing concentrations of analyte of interest, less aggregation of the complexes occur, and thus, the composition is less turbid.

In some embodiments, the detection particle 102 has a diameter ranging from about 0.001 microns (μm) to about 100 μm, such as 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 60, 70, 80, 90, or 100 μm, or a diameter within a range defined by any two of the aforementioned values. In some embodiments, the size of the detection particle is uniform for a plurality of detection particles, such that any one detection particle within the composition has a diameter that is identical or substantially identical to the diameter of every other detection particle in the composition, wherein the diameter is a size as described herein. In some embodiments, the complex 101 that includes a detection particle 102 and an immobilized analyte 103 is present in the composition in an amount sufficient to detect a trace amount of analyte of interest. In some embodiments, the amount of complex 101 in the composition ranges from 10 to 100,000,000 particles per mL, such as 10, 100, 1000, 2000, 3000, 4000, 5000, 6000, 7000, 8000, 9000, 10,000, 15,000, 20,000, 30,000, 40,000, 50,000, 60,000, 70,000, 80,000, 90,000, 100,000, 150,000, 200,000, 250,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, or 100,000,000, or an amount within a range defined by any two of the aforementioned values. In some embodiments, the quantity of complex in the composition is finely tuned to detect a quantity of target analyte, such that even in trace quantity of target analyte, decreased complex aggregation takes place, such that a detectable difference in agglomeration is detected.

In some embodiments, a quantity of immobilized analyte 103 present on the detection particle 102 is in an amount of 1 analyte per detection particle to 100,000 analyte per detection particle, such as 1, 2, 3, 4, 5, 6, 7, 8, 9, 10, 15, 20, 25, 30, 35, 40, 45, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, or 100,000 analyte compounds per detection particle, or an amount within a range defined by any two of the aforementioned values.

In some embodiments, the binding reagent is an antibody or a fragment thereof that specifically binds an analyte of interest. Thus, the antibody may be any antibody that specifically binds a specific drug (including toxic drugs, such as a hazardous drug including antineoplastic agents), a steroid, a toxin, a pesticide, a biowarfare agent, or any other detectable analyte of interest. In some embodiments, the binding reagent is present in the solution in an amount sufficient to cause aggregation of the detection particles, and is present in an amount sufficient to detect a quantity of analyte of interest in a test sample. The amount of binding reagent in the solution should be an amount such that a decrease in turbidity may be realized when an analyte of interest is present in the test sample. For example, if excess binding reagent is present, when analyte of interest is present in the test sample, excess binding reagent would still be present, causing the detection particles to aggregate, and thus, a quantity of analyte of interest may not be observable because no decrease in turbidity would be measured. Thus, the amount of detection reagent should be finely tuned to an amount that will result in a decrease in turbidity when analyte of interest is present in the test sample, including when trace amounts of analyte of interest is present in the test sample. Thus, for example, an amount of binding reagent should be an amount that is just sufficient to cause maximum turbidity in the absence of analyte of interest, but such that even in trace amounts of analyte of interest, a decrease in turbidity is observed due to inhibition of aggregation of the detection particles. Thus, in some embodiments, the amount of binding reagent present in the composition is in an amount ranging from 0.001 μg/ml to 10 μg/ml, such as 0.001, 0.005, 0.01, 0.05, 0.1, 0.5, 1, 2, 3, 4, 5, 6, 7, 8, 9, or 10 μg/ml, or an amount within a range defined by any two of the aforementioned values.

In some embodiments, the composition further includes aggregation enhancers, proteins, buffers, salts, or other reagents to enhance the ability of the complexes to aggregate in the absence of analyte of interest, or to decrease in aggregation in a predictable manner in the present of analyte of interest.

Measurement of turbidity may take place using any reader capable of measuring solution turbidity. Measuring changes in scattered light or apparent absorbance based on particle aggregation may be carried out using a turbidity meter, a spectrophotometer, or a detected that measures attenuation of light that impinged on a detector. Aggregation may be measured at wavelengths of light between 300 and 700 nm, such as at a wavelength of light of 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, or 700 nm, or at a wavelength of light within a range defined by any two of the aforementioned values. In the absence of analyte of interest, the aggregation of the particles results in a solution that scatters incident light and leads to an increase in the observed absorption of the solution. When a sample containing analyte of interest is introduced, the aggregation is partially inhibited, and binding reagent is no longer available to promote particle aggregation to the same extent as in the absence of analyte of interest, resulting in less scattering of incident light and lower observed absorption of the solution. In any of the embodiments described herein, the measurement of the turbidity of the composition may be used directly in the assay vessel, such as in a cuvette, a tube, a microtiter plate, or a microfluidic device, such that both the turbidimetric reaction and the turbidimetric measurement takes place in the same assay vessel. In some embodiments, the turbidimetric reaction takes place in one assay vessel, and after the reaction, the solution is transferred to a measurement assay, which is then placed in the reader. Multiplexing using a microtiter plate may be performed, for example, by providing a set of multiplexed assays arranged in a column of wells, and a single test sample could be applied to each well. Additional columns may be used for different samples for dilution measurements of analytes in order to generate a standard curve for the quantification of multiple analytes.

To quantify the amount of analyte of interest, a reference standard curve may be employed, wherein the standard curve provides a degree of turbidity that is inversely proportional to the concentration of the analyte of interest. Using a test sample, or a sample having an unknown concentration of analyte of interest, a degree of turbidity may be measured, and the degree of turbidity is compared to a reference standard curve to determine the concentration of the analyte of interest. In some embodiments, a standard curve may be a standard curve that is provided prior to measuring analyte concentration, and a measurement of the analyte of interest is compared to the provided standard curve to determine the concentration of analyte of interest. In some embodiments, a standard curve is determined at the time of measuring the analyte of interest based on generating a standard curve.

The systems and methods provided herein may be used to detect and determine the concentration of analyte of interest in trace amounts due to the sensitivity of the turbidimetric assay. Thus, for example, the systems and methods provided herein may be used to detect an analyte of interest having a concentration in the range of less than 10 ng/mL, less than 1 ng/mL, less than 100 pg/mL, less than 10 pg/mL, less than 1 pg/mL, less than 100 fg/mL, or less than 10 fg/mL, or an amount within a range defined by any two of the aforementioned values. In addition, the systems and methods provided herein may be used to detect and determine a concentration of analyte of interest in large quantities, including in amounts of more than 1 ng/mL, more than 10 ng/mL, more than 100 ng/mL, or more than 1 μg/mL. In some embodiments, where the concentration of analyte of interest is high, a sample comprising the analyte of interest may be diluted to decrease the effective concentration of analyte of interest in the measured assay. Thus, in some embodiments, the analyte of interest is present in an amount of 10, 15, 20, 25, 30, 35, 40, 50, 60, 70, 80, 90, 100, 200, 300, 400, 500, 600, 700, 800, 900, or 1000 fg/mL, or 1, 2, 3, 4, 5, 6, 7, 8, 9, 10, 15, 20, 25, 30, 35, 40, 50, 60, 70, 80, 90, 100, 200, 300, 400, 500, 600, 700, 800, 900, or 1000 pg/mL, or 1, 2, 3, 4, 5, 6, 7, 8, 9, 10, 15, 20, 25, 30, 35, 40, 50, 60, 70, 80, 90, 100, 200, 300, 400, 500, 600, 700, 800, 900, or 1000 ng/mL, or 1, 2, 3, 4, 5, 6, 7, 8, 9, or 10 μg/mL, or in an amount within a range defined by any two of the aforementioned values.

It is to be understood that the quantity of analyte of interest that is detected in the sample may be detected by finely tuning any one or more variables as described herein, such as by varying the size of the detection particle, the quantity of detection particle in the composition, the quantity of immobilized analyte bound to detection particle to form a complex, the quantity of complex in the composition, the quantity of binding reagent in the composition, or the wavelength of light at which the composition is monitored.

In some embodiments, the systems and assays allow for rapid determination of a presence and quantity of analyte of interest. For example, a measurement can be obtained by rapidly assessing the turbidity of a solution, wherein the turbidity reading can be obtained within minutes or seconds of contacting a sample with the assay composition. In some embodiments, an assay measurement can be obtained within a 1, 2, 3, 4, 5, 10, 15, 20, 25, 30, 35, 40, 45, 50, 55, or 60 seconds, or 1, 2, 3, 4, 5, 6, 7, 8, 9, or 10 minutes within contacting the sample with the assay composition, or in a time within a range defined by any two of the aforementioned values.

Although the disclosed detection devices are typically described herein with reference to turbidimetric assay measurements, it will be appreciated that the described hazardous contaminant detection system can be implemented in any suitable detection system. For example, features described herein can be implemented in reader devices that analyze other types of assays. Further, the collected fluid can be transferred to a centrifuge, spectrometer, chemical assay, lateral flow assay reader devices, or other suitable test device to determine the presence and/or concentration of one or more analyte of interest in the sample.

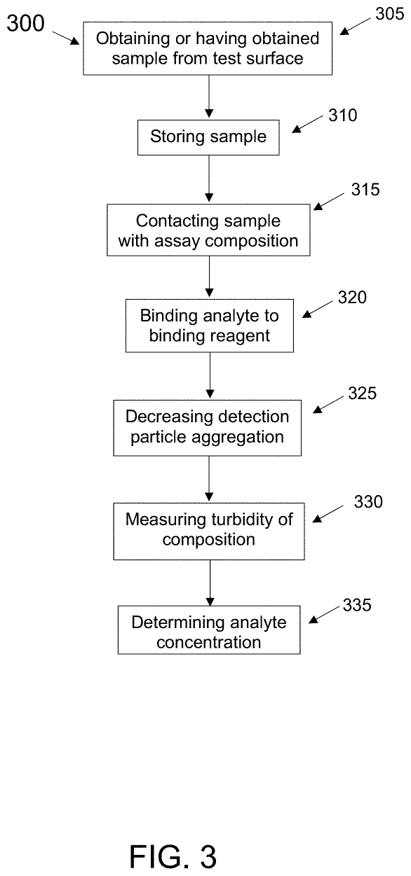

Some embodiments provided herein relate to methods of measuring a concentration of an analyte of interest using the turbidimetric systems described herein. With reference to FIG. 3, an exemplary method 300 is shown, wherein the method includes step 305, obtaining a sample from a test surface. In some embodiments, step 305 of obtaining a sample is performed by another, such that the sample is obtained, and then provided for subsequent analysis. Thus, step 305 can be performed by the person performing the measuring or analysis, or Step 305 may be performed by another, who provides the sample for analysis. Step 305 can be carried out using a collection device and kit as described herein, and transferring the collected sample to an assay vessel. In some embodiments, following step 305, the sample may be stored for a time in a storage step 310. In some embodiments, the storage step 310 is an optional step, wherein the collected sample is stored until the sample is ready to be analyzed. Step 315 includes contacting the sample with an assay composition. The assay composition can be any assay composition as described herein, wherein the composition includes reagents for conducting a turbidimetric assay, including a detection particle having immobilized analyte thereon and a binding reagent. Step 320 includes binding the analyte of interest to binding reagent. This binding reaction decreases the degree of turbidity in the solution as compared to a solution that does not have any analyte of interest present. Step 325 includes decreasing aggregation of the detection particle, which is due to the binding of binding reagent to analyte of interest, thereby inhibiting particle aggregation, and decreasing solution turbidity. Step 330 includes measuring the turbidity of the composition. The measurement may be carried out using any reader device as described herein. Step 335 includes determining the analyte concentration, which is determined based on the measured turbidity of the composition. The measured turbidity may be compared to a standard curve, which provides standard measurements of composition turbidity that is inversely proportional to analyte concentration.

Also provided herein are kits for carrying out a turbidity measurement. In some embodiments, the kits include a collection device, wherein the collection device is any collection device or kit as described herein. For example, the collection device may include a buffer solution configured to lift analyte of interest from a test surface when the buffer is applied to the test surface, an absorbent swab material configured to absorb at least a portion of the buffer solution and to contact the test surface to collect the analyte of interest, a handle having a first end coupled to the absorbent swab material, and a second end space apart from the first end, and an elongate length extending therebetween, and a fluid-tight container having an interior volume dimensioned to encase the handle and the absorbent swab material and the buffer solution, the container having a nozzle including an orifice sized to provide controlled release of a volume of the buffer solution from the interior volume. In some embodiments, the kits also include a turbidimetric composition as described herein. In some embodiments, the turbidimetric composition includes a detection particle comprising immobilized analyte and a binding reagent that specifically binds analyte of interest. The turbidimetric composition may be included in the kit in an assay vessel.

To use the kit, in some embodiments, the user employs the collection device on a test surface to collect the analyte of interest. In some embodiments, the user transfers the collected analyte of interest to the turbidimetric composition, which may be included in an assay vessel. In some embodiments, the user places the assay vessel in a reader device, or transfers the composition from the assay vessel to a measurement vessel, and then places the measurement vessel in the reader device. In some embodiments, the user obtains a measurement of turbidity of the composition. In some embodiments, the user compares the turbidity to a standard curve to determine the concentration of analyte of interest. In some embodiments, the standard curve is generated at the time, or close to the time, or measuring the analyte of interest, and the measured signal of turbidity obtained for the analyte of interest is compared to the standard curve. In some embodiments, the standard curve is a stored standard curve of turbidity measurements based on pre-determined concentrations of analyte, such that comparison to a standard curve is a mathematical comparison to a stored standard curve, or a pre-determined standard curve. In some embodiments, the reader automatically compares the measured turbidity to a standard curve, and generates a measure of analyte of interest. Thus, the reader is capable of outputting a measure of turbidity, a measure of the presence or absence of analyte of interest, or a measure of a concentration of analyte of interest.

### EXAMPLES

Embodiments of the present invention are further defined in the following Examples. It should be understood that these Examples are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims. The disclosure of each reference set forth herein is incorporated herein by reference in its entirety, and for the disclosure referenced herein.

### Example 1

### Measurement of Environmental Docetaxel

The following example demonstrates an example of measuring a quantity of docetaxel obtained from an environmental source using a turbidimetric assay.

Docetaxel is a commonly used antineoplastic agent of the taxoid family with broad activity in patients with a variety of solid tumors such as breast cancer, non-small cell lung cancer, hormone refractory prostate cancer, gastric adenocarcinoma, and squamous cell carcinoma of the head and neck. Docetaxel is a cell-cycle-specific cytotoxic agent, and is thus toxic to all cells in the body. Due to these effects, docetaxel is an occupational hazard with serious health effects for those working in hospitals or care facilities where this drug is prepared, administered, dispensed, or handled.

Docetaxel is obtained from an environmental source using a collection device as described herein. Briefly, docetaxel present in a hospital or pharmacy environment is obtained by contacting a test surface with a buffer solution. After incubating for a short period, the buffer solution on the test surface is absorbed on an absorbent swab material. The absorbent swab material is inserted into a fluid-tight container. The solution is then transferred to an assay vessel. The assay vessel includes latex particles having docetaxel immobilized thereon. The assay vessel also includes antibodies that specifically bind to docetaxel. Placement of the test sample in the assay vessel results in binding of the anti-docetaxel antibodies to docetaxel, and a decrease in turbidity as compared to a sample that does not have analyte of interest. The assay vessel is placed in a turbidity reader device, and a measurement of turbidity is compared to a standard curve to generate a measure of docetaxel concentration obtained from the test surface. The standard curve that is used, is a pre-determined standard curve that provides a measurement of turbidity for a known range of concentrations of an analyte of interest.

### Example 2

### Measurement of Environmental Paclitaxel

The following example demonstrates an example of measuring a quantity of paclitaxel obtained from an environmental source using a turbidimetric assay.

Paclitaxel is a commonly used antineoplastic agent of the taxoid family with broad activity in patients with a variety of solid tumors such as ovarian cancer, breast cancer, cervical cancer, pancreatic cancer, non-small cell lung cancer, and AIDS-related Kaposi's sarcoma. Paclitaxel is a cell-cycle-specific cytotoxic agent, and is thus toxic to all cells in the body. Due to these effects, paclitaxel is an occupational hazard with serious health effects for those working in hospitals or care facilities where this drug is prepared, administered, dispensed, or handled.

Paclitaxel is obtained from an environmental source using a collection device as described herein. Briefly, paclitaxel present in a hospital or pharmacy environment is obtained by contacting a test surface with a buffer solution. After incubating for a short period, the buffer solution on the test surface is absorbed on an absorbent swab material. The absorbent swab material is inserted into a fluid-tight container. The solution is then transferred to an assay vessel. The assay vessel includes latex particles having paclitaxel immobilized thereon. The assay vessel also includes antibodies that specifically bind to paclitaxel. Placement of the test sample in the assay vessel results in binding of the anti-paclitaxel antibodies to paclitaxel, and a decrease in turbidity as compared to a sample that does not have analyte of interest. The assay vessel is placed in a turbidity reader device, and a measurement of turbidity is compared to a standard curve to generate a measure of paclitaxel concentration obtained from the test surface. The standard curve that is used, is a pre-determined standard curve that provides a measurement of turbidity for a known range of concentrations of an analyte of interest.

### Example 3

### Measurement of Environmental Fluorouracil

The following example demonstrates an example of measuring a quantity of fluorouracil obtained from an environmental source using a turbidimetric assay.

Fluorouracil is a chemotherapeutic agent used in the treatment of several solid tumors cancers, such as colorectal, stomach, breast, pancreatic, and head and neck cancers. Fluorouracil inhibits DNA replication, and is thus toxic to all cells in the body. Due to these effects, fluorouracil is an occupational hazard with serious health effects for those working in hospitals or care facilities where this drug is prepared, administered, dispensed, or handled.

Fluorouracil is obtained from an environmental source using a collection device as described herein. Briefly, fluorouracil present in a hospital or pharmacy environment is obtained by contacting a test surface with a buffer solution. After incubating for a short period, the buffer solution on the test surface is absorbed on an absorbent swab material. The absorbent swab material is inserted into a fluid-tight container. The solution is then transferred to an assay vessel. The assay vessel includes latex particles having fluorouracil immobilized thereon. The assay vessel also includes antibodies that specifically bind to fluorouracil. Placement of the test sample in the assay vessel results in binding of the anti-fluorouracil antibodies to fluorouracil, and a decrease in turbidity as compared to a sample that does not have analyte of interest. The assay vessel is placed in a turbidity reader device, and a measurement of turbidity is compared to a standard curve to generate a measure of fluorouracil concentration obtained from the test surface. The standard curve that is used, is a pre-determined standard curve that provides a measurement of turbidity for a known range of concentrations of an analyte of interest.

The previous description of the disclosed implementations is provided to enable any person skilled in the art to make or use the present invention. Various modifications to these implementations will be readily apparent to those skilled in the art, and the generic principles defined herein may be applied to other implementations without departing from the spirit or scope of the invention. Thus, the present invention is not intended to be limited to the implementations shown herein but is to be accorded the widest scope consistent with the principles and novel features disclosed herein.

What is claimed is:

1. A method of quantifying an analyte of interest in a sample, comprising:
obtaining or having obtained a sample having or suspected of having an analyte of interest, wherein the sample is obtained from a test surface;
contacting the sample with a turbidimetric composition comprising:
a detection particle comprising immobilized analyte; and
a binding reagent that specifically binds analyte of interest;
measuring a turbidity of the composition;
wherein the measured turbidity corresponds to a quantity of analyte of interest in the sample.

2. The method of claim 1, wherein obtaining the sample form the test surface comprises:
collecting the analyte of interest from the test surface;
placing the collected analyte of interest in a test solution;
removing an aliquot of the test solution; and
transferring the aliquot to an assay vessel comprising the turbidimetric composition.

3. The method of claim 2, wherein the assay vessel is a cuvette, a tube, a microtiter plate, or a microfluidic device.

4. The method of claim 1, wherein obtaining the sample from the test surface comprises:
wiping the test surface with an absorbent material to collect the analyte of interest from the test surface, wherein the absorbent material is moistened with a first volume of a buffer solution configured to lift the analyte of interest from the test surface;
inserting the absorbent material into a collection container, the collection container comprising a second volume of the buffer solution;
sealing the collection container to isolate the first and second volumes of the buffer solution within the collection container;
agitating the collection container to release at least some of the collected analyte of interest into the buffer solution; and
transferring a third volume of the buffer solution from the collection container to an assay vessel comprising the turbidimetric composition.

5. The method of claim 4, wherein the absorbent material is coupled to an elongate handle.

6. The method of claim 1, wherein the detection particle aggregates with other detection particles present in the turbidimetric composition in the absence of analyte of interest, thereby increasing turbidity of the turbidimetric composition, and wherein increased concentrations of analyte of interest in the sample decreases aggregation of the detection particles, thereby decreasing turbidity of the turbidimetric composition.

7. The method of claim 1, wherein the turbidity of the turbidimetric composition is measured using a detection device that measures turbidity of the turbidimetric composition.

8. The method of claim 7, wherein the detection device comprises control electronics configured to analyze signals representative of analyte concentration and configured to determine the quantity of the analyte of interest in the sample based on turbidity of the turbidimetric composition.

9. The method of claim 1, wherein the measured turbidity of the turbidimetric composition is compared to a standard curve to determine the quantity of the analyte of interest.

10. The method of claim 1, wherein the analyte of interest is cyclophosphamide, docetaxel, fluorouracil, ifosfamide, imatinib, or paclitaxel.

11. The method of claim 1, wherein the test surface is a counter, a cabinet, an instrument, a bed, a chair, a table, a floor, a window, a toilet, or a wall.

12. The method of claim 1, wherein the analyte of interest is not obtained from a plasma sample.

13. The method of claim 1, wherein the detection particle comprises a nanoparticle, organic or inorganic polymers, liposomes, latex, phospholipid vesicles, or lipoproteins.

14. The method of claim 1, wherein the binding reagent is an antibody or fragment thereof that specifically binds the analyte of interest.

15. The method of claim 1, wherein the analyte of interest is present in an amount ranging from 10 fg/mL to 10 μg/mL.

16. The method of claim 1, wherein the detection particle is present in an amount ranging from 10 to 100,000,000 particles per mL.

17. The method of claim 1, wherein the immobilized analyte is present on the detection particle in an amount ranging from 1 analyte per detection particle to 100,000 analyte per detection particle.

18. The method of claim 1, wherein the binding reagent is present in an amount ranging from 0.001 μg/mL to 10 μg/mL.

19. The method of claim 1, wherein measuring the turbidity of the turbidimetric composition is performed at a wavelength ranging from 300 to 700 nm.

20. A kit for determining a quantity of an analyte of interest obtained from a test surface, wherein the kit comprises:
an analyte collection device for collecting the analyte of interest from a test surface;
a turbidimetric composition comprising:
a detection particle comprising immobilized analyte; and
a binding reagent that specifically binds analyte of interest, and
an assay vessel.
